(19) United States
(12) Patent Application Publication
(10) Pub. No.: US 2022/0151707 A1
(43) Pub. Date: May 19, 2022

(54) PUNCTURE INFORMATION PROCESSING DEVICE, ULTRASONIC LAPAROSCOPIC PUNCTURING SYSTEM, PUNCTURE INFORMATION PROCESSING METHOD, AND STORAGE MEDIUM

(71) Applicant: CANON MEDICAL SYSTEMS CORPORATION, Otawara-shi (JP)

(72) Inventors: Shohei SASAKI, Otawara (JP); Tomohiro SATO, Otawara (JP); Yasushi KAMEWADA, Otawara (JP)

(73) Assignee: CANON MEDICAL SYSTEMS CORPORATION, Otawara-shi (JP)

(21) Appl. No.: 17/525,193

(22) Filed: Nov. 12, 2021

(30) Foreign Application Priority Data
Nov. 17, 2020 (JP) 2020-190953

(51) Int. Cl. A61B 34/20; A61B 8/08; A61B 8/00; A61B 8/12

(52) U.S. Cl. CPC A61B 34/20; A61B 8/0841; A61B 2034/2063; A61B 8/12; A61B 8/42

(57) ABSTRACT

A puncture information processing device of an embodiment includes processing circuitry. The processing circuitry is configured to acquire position information on a passage through which a puncture needle for puncturing a living body passes when an ultrasonic probe provided with the passage is inserted into the living body, and derive a body surface puncture position on a body surface of the living body to be punctured with the puncture needle passing through the passage on the basis of the acquired position information on the passage.

### CROSS-REFERENCE TO RELATED APPLICATION

The present application claims priority based on Japanese Patent Application No. 2020-190953, filed Nov. 17, 2020, the content of which is incorporated herein by reference.

### FIELD

Embodiments disclosed in the present description and drawings relate to a puncture information processing device, an ultrasonic laparoscopic puncturing system, a puncture information processing method, and a storage medium.

### BACKGROUND

Laparoscopic surgery is an operation performed by making a plurality of small holes around a surgical site and inserting a surgical instrument and a diagnostic instrument such as an ultrasonic probe into the body through a tubular member called a trocar. The tip of an ultrasonic probe used in laparoscopic surgery is provided with, for example, a puncture hole or groove, and an organ can be punctured at any angle by passing a puncture needle through the puncture hole or groove of the ultrasonic probe.

### BRIEF DESCRIPTION OF THE DRAWINGS

FIG. 1 is a diagram showing an example of a configuration of an ultrasonic laparoscopic puncturing system 1 of a first embodiment.

FIG. 2 is a cross-sectional view of a state in which an operation or the like is being performed using the ultrasonic laparoscopic puncturing system 1.

FIG. 3 is a plan view of an ultrasonic probe 10.

FIG. 4 is a flowchart showing an example of processing of a puncture information processing device 100.

FIG. 5 is a diagram showing an example of a configuration of an ultrasonic laparoscopic puncturing system 2 of a second embodiment.

FIG. 6 is a cross-sectional view of a state in which an operation or the like is performed using the ultrasonic laparoscopic puncturing system 2.

FIG. 7A is a diagram showing an example of puncture information displayed on a display device 40.

FIG. 7B is a diagram showing an example of puncture information displayed on the display device 40.

FIG. 8 is a diagram showing an example of a configuration of an ultrasonic laparoscopic puncturing system 3 of a third embodiment.

FIG. 9 is a cross-sectional view of a state in which an operation or the like is performed using the ultrasonic laparoscopic puncturing system 3.

### DETAILED DESCRIPTION

In a puncturing procedure using an ultrasonic probe for laparoscopic surgery, the ultrasonic probe is inserted from a trocar and a puncture needle is punctured directly from the body surface and passed through a hole or groove provided in the ultrasonic probe, in general. Therefore, if a body surface puncture position is not appropriate, there are some cases where it is not possible to pass the puncture needle through the hole or groove of the ultrasonic probe. A puncture information processing device of an embodiment includes processing circuitry. The processing circuitry is configured to acquire position information on a passage through which a puncture needle for puncturing a living body passes when an ultrasonic probe provided with the passage is inserted into the living body, and derive a body surface puncture position on a body surface of the living body to be punctured with the puncture needle passing through the passage on the basis of the acquired position information on the passage.

Hereinafter, a puncture information processing device, an ultrasonic laparoscopic puncturing system, a puncture information processing method, and a storage medium of embodiments will be described with reference to the drawings.

### First Embodiment

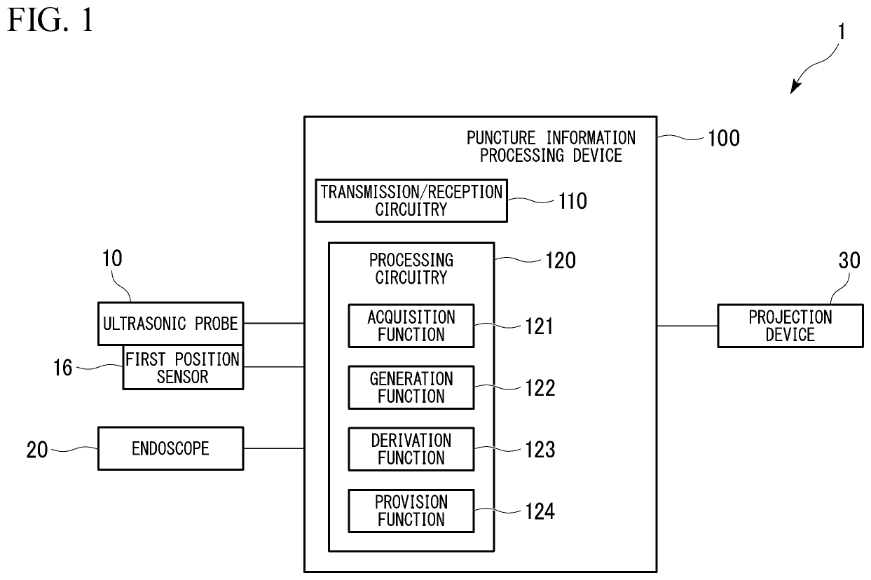

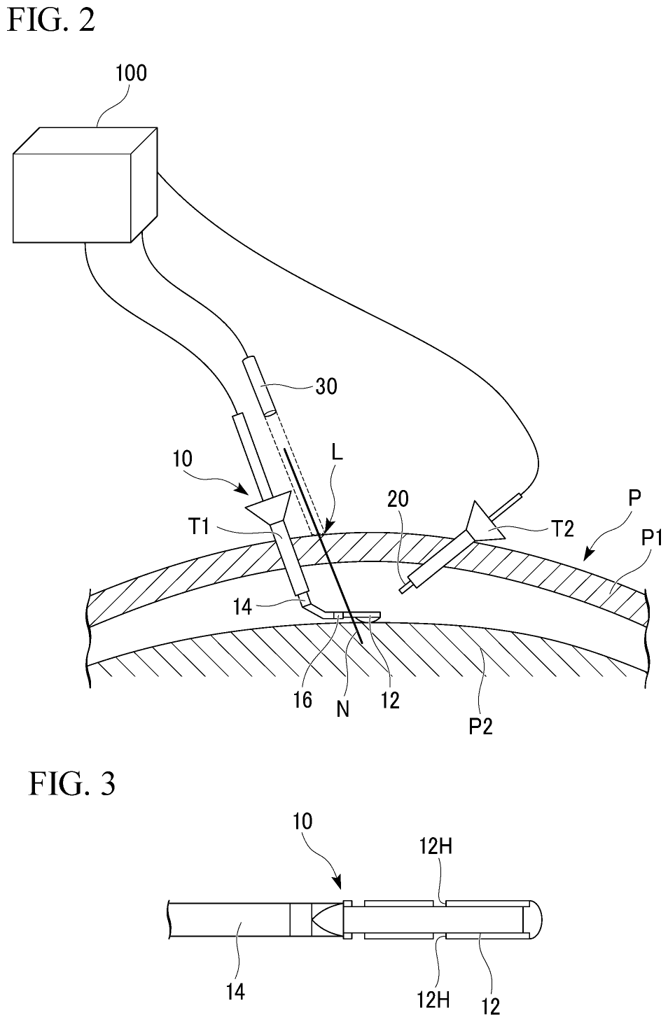

FIG. 1 is a diagram showing an example of a configuration of an ultrasonic laparoscopic puncturing system 1 of the first embodiment. FIG. 2 is a cross-sectional view of a state in which an operation or the like is performed using the ultrasonic laparoscopic puncturing system 1. FIG. 3 is a plan view of an ultrasonic probe 10. The ultrasonic laparoscopic puncturing system 1 includes, for example, an ultrasonic probe 10, an endoscope 20, a projection device 30, and a puncture information processing device 100.

An operator such as a doctor or a laboratory technician punctures a patient P with a puncture needle N in order to treat an affected area of the patient P, and the like, for example, when performing an operation or an examination (hereinafter referred to as an “operation or the like”) of a living body, for example, the patient P. The ultrasonic laparoscopic puncturing system 1 is a device that provides the operator with a puncture position (hereinafter referred to as a “body surface puncture position”) of the puncture needle N on the body surface of the patient P when the operator punctures the puncture needle N. The position of the affected area (hereinafter, referred to as an “affected area position”) is, for example, an arrival point that is an arrival target of the puncture needle N.

The ultrasonic probe 10 includes, for example, a transmission/reception head 12, a supporter 14, and a first position sensor 16. The ultrasonic probe 10 is used, for example, to acquire an ultrasonic image in the body of patient P. The ultrasonic probe 10 is also used to identify the position of the affected area in the body of the patient P. It is assumed that an affected area position is inside an internal organ of the patient P in the following description.

The transmission/reception head 12 is arranged, for example, between the skin P1 and an internal organs P2 of the patient P at the time of performing an operation or the like of the patient P. The transmission/reception head 12 transmits ultrasonic waves and receives reflected waves of the transmitted ultrasonic waves. The ultrasonic probe 10 generates reflected wave information based on the received reflected waves of the ultrasonic waves and transmits the reflected wave information to the puncture information processing device 100.

A puncture groove 12H is provided at a lateral position of the transmission/reception head 12 in a plan view. The puncture groove 12H is a groove that guides the position of the puncture needle N when the puncture needle N punctures an internal organ of a patient. Although one puncture groove 12H is provided on each side of the transmission/reception head 12, two or more puncture grooves 12H may be provided on one side or both sides of the transmission/reception head 12. The puncture groove 12H may be provided on a portion other than both sides of the transmission/reception head 12, or may be provided at a position in the supporter 14 disposed between the skin P1 and the internal organ P2 of the patient P at the time of performing an operation or the like.

The supporter 14 is a long member having a transmission/reception head 12 attached to the tip thereof. The transmission/reception head 12 of the ultrasonic probe 10 is introduced between the skin P1 and the internal organ P2 of the patient P from the outside of the patient P through the inside of the first trocar T1 at the time of performing an operation or the like. When the transmission/reception head 12 is arranged between the skin P1 and the internal organ P2, the supporter 14 has passed through the first trocar T1. The supporter 14 is rotatable in the first trocar T1. By rotating the supporter 14 in the first trocar T1, the transmission/reception head 12 can adjust the orientation thereof between the skin P1 and the internal organ P2.

The first position sensor 16 is built into the supporter 14, for example, at the end of the supporter 14 on the side where the transmission/reception head 12 is provided. The first position sensor 16 may be provided on the transmission/reception head 12. The first position sensor 16 may be provided in the vicinity of an operating part of the ultrasonic probe 10. The operating part of the ultrasonic probe 10 is a part operated by the operator and is provided on the operator side of the first trocar T1 when the transmission/reception head 12 passes through the first trocar T1 and is arranged between the skin P1 and the internal organ P2 of the patient P.

When the first position sensor 16 is provided on the transmission/reception head 12 or the like, it is desirable that the first position sensor 16 be small enough to pass through the first trocar T1. When the first position sensor 16 is arranged on the operating part side of the first trocar T1 such as the operating part, the size of the first position sensor 16 is not limited as much as when provided on the transmission/reception head 12.

When the first position sensor 16 is provided at a position other than the transmission/reception head 12, the position of the puncture groove 12H may be obtained by correcting the position detected by the first position sensor 16 using a relative positional relationship between the puncture groove 12H and the first position sensor 16. When the supporter 14 of the ultrasonic probe 10 is deformed, the position of the puncture groove 12H may be obtained by correcting the position detected by the first position sensor 16 in consideration of the deformation of the supporter 14.

As the first position sensor 16, for example, a 6-axis sensor can be used. As the 6-axis sensor, a sensor that detects inertial forces of a total of 6 axes including accelerations in three dimensional directions of X-Y-Z axes and the angular velocity of each axis can be used. Instead of the 6-axis sensor, two 3-axis sensors, for example, a combination of a 3-axis sensor that detects accelerations in three dimensional directions and a 3-axis sensor that detects the angular velocity of each axis may be used. In general, a 3-axis sensor is smaller than a 6-axis sensor. Therefore, when the first position sensor 16 is provided, for example, on the transmission/reception head 12 that needs to be passed through the first trocar T1, the transmission/reception head 12 can be miniaturized by using the aforementioned two 3-axis sensors.

The first position sensor 16 detects the position of the puncture groove 12H in the transmission/reception head 12. When the ultrasonic probe 10 is inserted into the body, the puncture needle N passes through the puncture groove 12H toward an affected area position. The puncture groove 12H is an example of a passage. In addition to the groove, the passage may be a hole provided in the transmission/reception head 12 or the like of the ultrasonic probe 10. The first position sensor 16 transmits the detected positions of the transmission/reception head 12 and the puncture groove 12H to the puncture information processing device 100.

The endoscope 20 is a small-diameter device provided with a camera at the tip thereof. The endoscope 20 is introduced between the skin P1 and the internal organ P2 of the patient P through the inside of a second trocar T2 which is different from the first trocar T1, for example, at the time of performing an operation or the like. The endoscope 20 images, for example, the transmission/reception head 12 of the ultrasonic probe 10 and the puncture needle N. The endoscope 20 transmits a captured image to the puncture information processing device 100.

The projection device 30 is disposed, for example, at a position where light can be radiated to the body surface of the patient P. The projection device 30 is a laser pointer that radiates light that specifies a certain region, for example, laser light L. The projection device 30 projects light by radiating the laser light L to a body surface puncture position. For example, when the body surface puncture position is in a shadow of the ultrasonic probe 10, the endoscope 20, the first trocar T1, and the second trocar T2, the projection device 30 is provided in a moving device that is not shown such that it can change the position. The projection device 30 may be provided at a fixed position. Two or more projection devices 30 may be provided such that the laser light L can be radiated from different directions. The projection device 30 may be other than a laser pointer. For example, the projection device 30 may be a projector such as a projector that projects an image of 3D mapping, or a projector such as a projector that projects an image of projection mapping. The projection device 30 is an example of a projector.

The puncture information processing device 100 includes, for example, transmission/reception circuitry 110 and processing circuitry 120. The transmission/reception circuitry 110 includes, for example, a drive circuit for transmitting ultrasonic waves to the transmission/reception head 12 of the ultrasonic probe 10, and the like. The transmission/reception circuitry 110 outputs a drive signal to the ultrasonic probe 10 via a cable according to transmission/reception conditions transmitted by the processing circuitry 120. The transmission/reception circuitry 110 acquires reflected wave information output from the ultrasonic probe 10. The transmission/reception circuitry 110 converts the acquired reflected wave information into a digital signal and outputs the digital signal to the processing circuitry 120.

The processing circuitry 120 includes, for example, an acquisition function 121, a generation function 122, a derivation function 123, and a provision function 124. The processing circuitry 120 realizes these functions by, for example, a hardware processor executing a program stored in a memory (memory circuit). For example, the hardware processor means a circuit (circuitry) such as a central processing unit (CPU), a graphics processing unit (GPU), an application specific integrated circuit (ASIC), and a programmable logic device (for example, a simple programmable logic device (SPLD) and a complex programmable logic device (CPLD)), and a field programmable gate array (FPGA), etc. (the same applies hereinafter). The program may be directly embedded in the circuit of the hardware processor instead of being stored in the memory. In this case, the hardware processor realizes a function by reading and executing the program embedded in the circuit. The hardware processor is not limited to a single circuit and may be configured as one hardware processor by combining a plurality of independent circuits to realize each function. Further, a plurality of components may be integrated into one hardware processor to realize each function. The memory is realized by, for example, a random access memory (RAM), a semiconductor memory element such as a flash memory, a hard disk, an optical disc, or the like.

The acquisition function 121 acquires various types of information output from the transmission/reception circuitry 110 and outputs the information to the generation function 122. The information acquired by the acquisition function 121 includes, for example, reflected wave information output from the ultrasonic probe 10 and information on the position of the puncture groove 12H in the transmission/reception head 12 output from the first position sensor 16. The acquisition function 121 acquires, for example, position information of the puncture groove 12H when the ultrasonic probe 10 has been inserted into the body of the patient P. The acquisition function 121 outputs the reflected wave information of the acquired information to the generation function 122 and outputs the position information of the puncture groove 12H to the derivation function 123. In this manner, the acquisition function 121 acquires position information of the passage of the puncture groove 12H when the ultrasonic probe 10 provided with the puncture groove 12H through which the puncture needle N passes has been inserted into the body of the patient P. The acquisition function 121 is an example of an acquirer.

The generation function 122 generates an ultrasonic image on the basis of the reflected wave information output from the acquisition function 121. The generation function 122 outputs the generated ultrasonic image to the derivation function 123. The derivation function 123 identifies an affected area position on the basis of the ultrasonic image output from the generation function 122.

The derivation function 123 generates a puncture guideline on the basis of the identified affected area position and the position of the puncture groove 12H output from the acquisition function 121. The derivation function 123 derives a body surface puncture position on the basis of the identified affected area position and the generated puncture guideline. The derivation function 123 may derive the body surface puncture position on the basis of the position of the puncture groove 12H and the affected area position. The derivation function 123 outputs the derived body surface puncture position to the provision function 124.

A shape formed by the puncture guideline is determined according to the puncture needle N. For example, the derivation function 123 derives a linear puncture guideline when the puncture needle N is linear and derives a puncture guideline having a curvature matching the curvature of the puncture needle N when the puncture needle N is curved. In this manner, the derivation function 123 derives the body surface puncture position on the body surface of the patient P at which the puncture needle N passing through the puncture groove 12H is punctured on the basis of the position of the puncture groove 12H. The derivation function 123 is an example of a deriver.

The provision function 124 outputs the body surface puncture position derived by the derivation function 123 to the projection device 30 and causes the projection device 30 to project light by radiating the laser light L to the body surface puncture position. The provision function 124 provides the operator with information on the body surface puncture position as information on the body surface puncture position by causing the projection device 30 to radiate the laser light L to the body surface puncture position. The provision function 124 is an example of a provider.

Next, a flow in which the operator punctures the puncture needle N using the ultrasonic laparoscopic puncturing system 1 of the first embodiment will be described. For example, in treating an affected area, the operator introduces the transmission/reception head of the ultrasonic probe 10 into the body of the patient P between the skin and internal organs of the subject. Subsequently, the operator punctures the patient P with the puncture needle N to treat the affected area. When the operator treats the affected area, the puncture information processing device 100 executes a process of providing the operator with a body surface puncture position to inform the operator of the body surface puncture position.

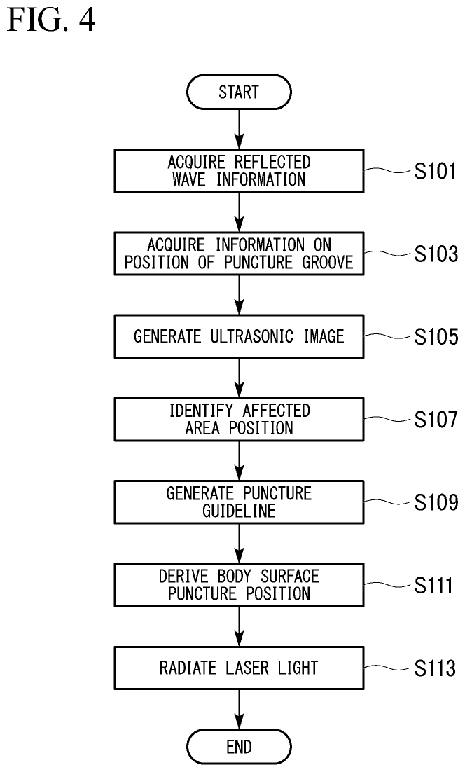

FIG. 4 is a flowchart showing an example of processing of the puncture information processing device 100. First, the puncture information processing device 100 causes the ultrasonic probe 10 to transmit ultrasonic waves. The ultrasonic probe 10 receives reflected waves of the transmitted ultrasonic waves, generates reflected wave information, and outputs the reflected wave information to the puncture information processing device 100.

The puncture information processing device 100 receives a reflected wave signal output from the ultrasonic probe 10 in the acquisition function 121 (step S101). Further, the first position sensor 16 outputs information on a detected position of the puncture groove 12H in the ultrasonic probe 10 to the puncture information processing device 100 (step S103).

Subsequently, the generation function 122 generates an ultrasonic image on the basis of ultrasonic information output from the ultrasonic probe 10 (step S105) and outputs the generated ultrasonic image to the derivation function 123. The derivation function 123 analyzes the ultrasonic image output from the generation function 122 to identify the position of the affected area (step S107). The ultrasonic image generated by the generation function 122 may be displayed by a display device (not shown). The affected area position may be identified by the operator or the like via an input interface, for example. As the input interface, for example, a mouse or a keyboard may be used.

In the present description, the input interface is not limited to the one provided with physical operating parts such as a mouse and a keyboard. For example, examples of an input interface may include electrical signal processing circuitry that receives an electrical signal corresponding to an input operation from an external input device provided separately from the device and outputs the electrical signal to control circuitry.

Subsequently, the derivation function 123 generates a puncture guideline on the basis of the identified affected area position and the position information of the puncture groove 12H output from the acquisition function 121 (step S109). After generating the puncture guideline, the derivation function 123 derives a body surface puncture position on the basis of the generated puncture guideline (step S111). The body surface puncture position is, for example, a point where the puncture guideline and the body surface of patient P intersect.

The provision function 124 causes the projection device 30 to radiate the laser light L so as to illuminate the body surface puncture position derived by the derivation function 123 with the laser light L. The provision function 124 radiates the laser light L to the body surface puncture position to provide the body surface puncture position to the operator such that the operator is informed of the body surface puncture position according to the radiated laser light L (step S113). In this manner, the puncture information processing device 100 ends processing shown in FIG. 4.

The operator who is provided with the body surface puncture position by the puncture information processing device 100 causing the projection device 30 to radiate the laser light to the body surface puncture position punctures the body surface puncture position with the puncture needle N. Therefore, the operator can easily and accurately puncture the body surface puncture position with the puncture needle N.

In the ultrasonic laparoscopic puncturing system 1 of the first embodiment, when the operator punctures the patient P with the puncture needle N, the body surface puncture position is irradiated with the laser light L, and the body surface puncture position is provided to the operator such that the operator is informed of the body surface puncture position. Therefore, the operator can easily recognize the position where the puncture needle N is to be punctured, and a puncturing procedure can be facilitated by using the ultrasonic probe for laparoscopic surgery.

The ultrasonic laparoscopic puncturing system 1 derives the body surface puncture position using the puncture groove 12H detected by the first position sensor 16 provided on the ultrasonic probe 10 and the puncture guideline generated on the basis of affected area position. Accordingly, an appropriate body surface puncture position can be obtained. Further, the body surface puncture position can be ascertained regardless of the position of the tip of the ultrasonic probe 10. Therefore, it is possible to improve the quality of an image displaying the body surface puncture position.

### Second Embodiment

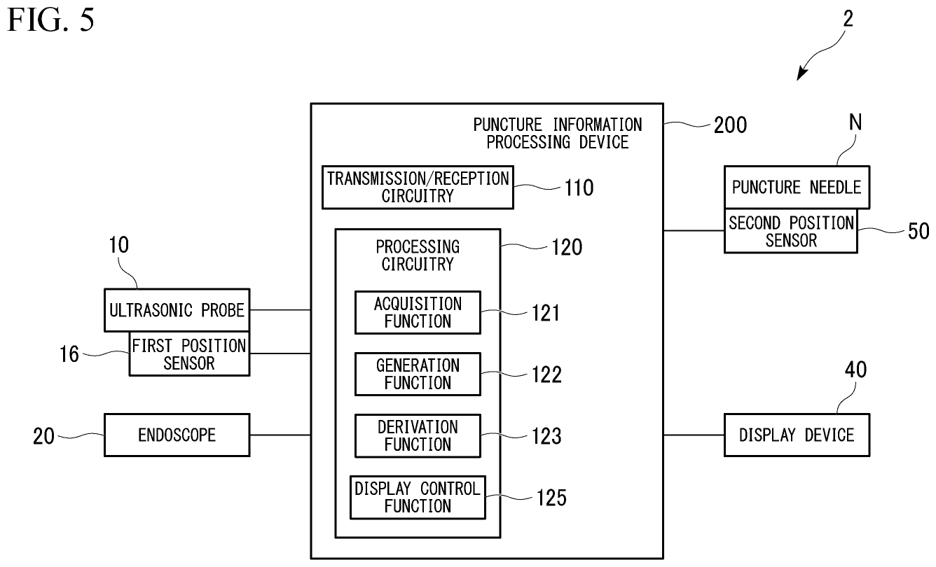

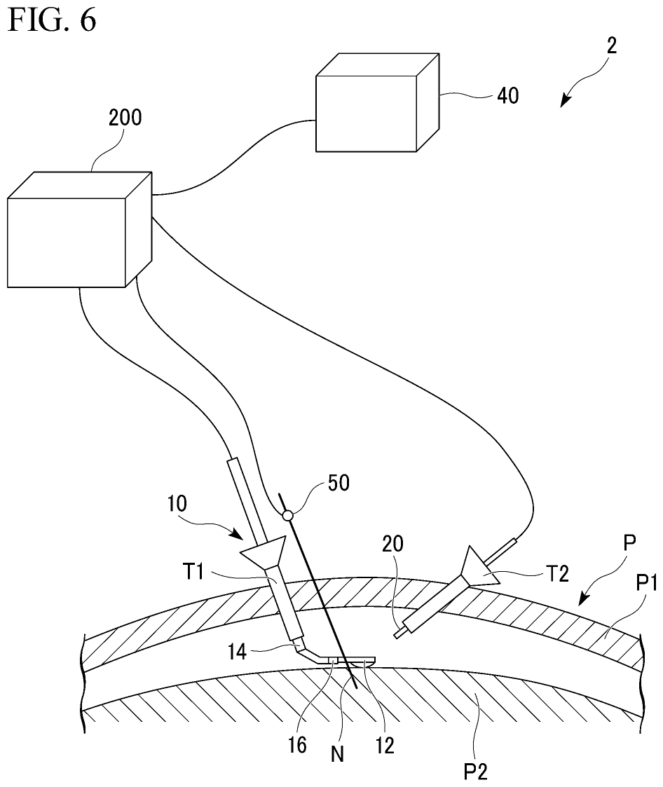

Next, a second embodiment will be described. FIG. 5 is a diagram showing an example of a configuration of an ultrasonic laparoscopic puncturing system 2 of the second embodiment and FIG. 6 is a cross-sectional view of a state in which an operation or the like is performed using the ultrasonic laparoscopic puncturing system 2. The ultrasonic laparoscopic puncturing system 2 of the second embodiment differs from the ultrasonic laparoscopic puncturing system 1 of the first embodiment in that the former includes a display device 40 instead of the projection device 30, a display control function 125 instead of the provision function 124 in the processing circuitry 120, and a second position sensor 50 in the processing circuitry 120. Hereinafter, the ultrasonic laparoscopic puncturing system 2 of the second embodiment will be described focusing on the differences from the ultrasonic laparoscopic puncturing system 1 of the first embodiment.

The display device 40 is, for example, a liquid crystal display. The display device 40 may be any device that displays an image and may be, for example, a projector. When the display device 40 is a projector, the display device 40 may project an image on a screen or a three-dimensional space. The display device 40 may be VR goggles worn by the operator. The display device 40 is an example of a display.

The display device 40 displays, for example, information on the position and angle when the puncture needle N is punctured (hereinafter, referred to as “position angle information”), and the like according to control of the display control function 125. The display device 40 is arranged at a position that can be visually recognized by the operator during an operation, for example. In addition, the display device 40 may display an ultrasonic image based on reflected waves received by the ultrasonic probe 10 or an image captured by the endoscope 20.

The second position sensor 50 is built into the puncture needle N, for example. The second position sensor 50 detects the position of the needle tip of the puncture needle N (hereinafter referred to as “needle tip position”) and the puncture angle of the puncture needle N. The second position sensor 50 transmits information on the detected needle tip position and puncture angle to a puncture information processing device 200. The second position sensor 50 is, for example, a 6-axis sensor. The second position sensor 50 may be, for example, a combination of a 3-axis sensor that detects accelerations in a three-dimensional direction and a 3-axis sensor that detects the angular velocity of each axis. The second position sensor 50 transmits the information on the detected needle tip position and puncture angle to the puncture information processing device 200.

Here, the puncture angle will be described. For example, an XY plane that includes a body surface puncture position in any XYZ space and has Z=0 is defined. The puncture angle includes, for example, a puncture angle around the X-axis and a puncture angle around the Y-axis. The puncture angle around the X-axis is an angle at which a straight line in the extending direction of the puncture needle N has rotated around the X-axis from a reference line, for example, when the puncture needle N is translated to the body surface puncture position on the XY plane. The puncture angle around the Y-axis is an angle at which the straight line in the extending direction of the puncture needle N has rotated around the Y-axis from the reference line, for example, when the puncture needle N is translated to the body surface puncture position on the XY plane.

The puncture information processing device 200 acquires the information on the needle tip position and puncture angle transmitted from the second position sensor 50 through the acquisition function 121. As in the first embodiment, the generation function 122 generates an ultrasonic image on the basis of the reflected wave information, and the derivation function 123 generates a puncture guideline on the basis of the affected area position and the position of the puncture groove 12H.

Further, the derivation function 123 calculates a rotation angle of the puncture guideline (hereinafter referred to as a “guideline angle”). The guideline angle includes, for example, a guideline angle around the X-axis and a guideline angle around the Y-axis. The guideline angle around the X-axis is, for example, an angle at which the puncture guideline has rotated around the X-axis from a reference line. The guideline angle around the Y-axis is, for example, an angle at which the puncture guideline has rotated around the Y-axis from the reference line.

The derivation function 123 derives position angle information. The position angle information includes information on an amount x of deviation in the X direction, an amount y of deviation in the Y direction, an amount θ of deviation around X axis, and an amount φ of deviation around the Y axis. The derivation function 123 derives the amount x of deviation in the X direction and the amount y of deviation in the Y direction on the basis of the needle tip position and the derived body surface puncture position and derives the amount θ of deviation around the X axis and the amount φ of deviation around the Y axis on the basis of the puncture angle and the guideline angle. The amount x of deviation in the X direction, the amount y of deviation in the Y direction, the amount θ of deviation around the X axis, and the amount φ of deviation around the Y axis are examples of a relative relationship of the needle tip position with respect to the body surface puncture position.

The amount x of deviation in the X direction is, for example, the amount of deviation (length) in the X direction between the needle tip position and the body surface puncture position on the XY plane. The amount y of deviation in the Y direction is, for example, the amount of deviation (length) in the Y direction between the needle tip position and the body surface puncture position on the XY plane. The amount θ of deviation around the X axis is the amount of deviation (angle) of the puncture angle and the guideline angle around the X-axis. The amount y of deviation around the Y axis is the amount of deviation (angle) of the puncture angle and the guideline angle around the Y-axis.

The derivation function 123 outputs the derived position angle information to the display control function 125. The display control function 125 controls the display device 40 such that the display device 40 displays an image representing the position angle information output from the derivation function 123.

Next, a case where the operator punctures the puncture needle N using the ultrasonic laparoscopic puncturing system 2 of the second embodiment will be described. In this case, the puncture information processing device 200 executes the same processing as processing from acquisition of the reflected wave information in step S101 to derivation of the body surface puncture position in step S111 in FIG. 4.

Subsequently, the puncture information processing device 200 derives position angle information in the derivation function 123, and the display control function 125 causes the display device 40 to display the position angle information derived by the derivation function 123, for example, using numerical values or a figure. An example of position angle information displayed on the display device 40 by the display control function 125 will be described.

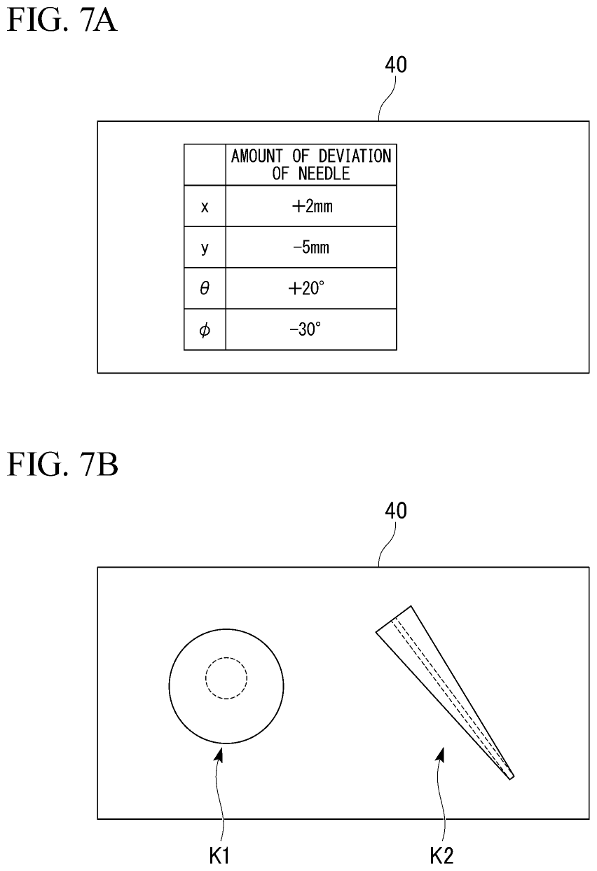

FIG. 7A and FIG. 7B are diagrams showing examples of position angle information displayed on the display device 40. For example, the display control function 125 displays position angle information using numerical values as shown in FIG. 7A. In the example shown in FIG. 7A, the display control function 125 causes the display device 40 to display +2 mm as the amount x of deviation in the X direction and −5 mm as the amount y of deviation in the Y direction. Further, the display control function 125 causes the display device 40 to display +20° as the amount θ of deviation around the X axis and −30° as the amount φ of deviation around the Y axis.

The operator adjusts a needle tip position and a puncture angle by visually recognizing these displays. When the needle tip position and the puncture angle are adjusted, the position angle information is updated at any time. The operator adjusts the needle tip position and the puncture angle by moving the puncture needle N until each item of the position angle information becomes 0 while observing the updated position angle information. The operator starts puncturing the puncture needle N when each item of the position angle information becomes 0. Accordingly, the operator can easily adjust the position and angle of the puncture needle N to the body surface puncture position and the angle of the puncture guideline.

Alternatively, as shown in FIG. 7B, the display control function 125 causes the display device 40 to display the position angle information using a figure. In the example shown in FIG. 7B, the amount of deviation between the position of the puncture needle N and the body surface puncture position is represented by a first information figure K1 on the left side and a deviation between the angle of the puncture needle N and the angle of the puncture guideline is represented by a second information figure K2 on the right side.

In the first information figure K1, the amount of deviation between the position of the puncture needle N and the body surface puncture position is expressed by a difference between circle sizes. The circle represented by a broken line in the first information figure K1 is displayed when the position of the puncture needle N matches the body surface puncture position. The circle represented by a solid line in the first information figure K1 is displayed when the position of the puncture needle N and the body surface puncture position are deviated from each other. The larger the circle displayed as represented by the solid line, the larger the amount of deviation between the position of the puncture needle N and the body surface puncture position.

The display control function 125 may display only the circle indicating the amount of deviation between the position of the puncture needle N and the body surface puncture position, as represented by the solid line, as the first information figure K1. The display control function 125 may display the circle indicating that the position of the puncture needle N matches the body surface puncture position, represented by the broken line, in addition to the circle indicating the amount of deviation between the position of the puncture needle N and the body surface puncture position, represented by the solid line.

In the second information figure K2, the amount of deviation between the angle of the puncture needle N and the angle of the puncture guideline is expressed using a substantially isosceles triangle having an inner rectangle as a center. The rectangle represented by the broken line in the second information figure K2 is displayed when the angle of the puncture needle N matches the angle of the puncture guideline. The substantially isosceles triangle represented by a solid line in the second information figure K2 is displayed when the angle of the puncture needle N and the angle of the puncture guideline deviate from each other. The larger the angle between the equal sides of the substantially isosceles triangle displayed as represented by the solid line, the larger the amount of deviation between the angle of the puncture needle N and the angle of the puncture guideline.

The display control function 125 may display only the substantially isosceles triangle indicating the amount of deviation between the angle of the puncture needle N and the angle of the puncture guideline, as represented by the solid line, as the second information figure K2. The display control function 125 may display the rectangle indicating that the angle of the puncture needle N matches the angle of the puncture guideline, represented by the broken line, in addition to the substantially isosceles triangle indicating the amount of deviation between the angle of the puncture needle N and the angle of the puncture guideline.

In this manner, the display control function 125 can appeal to the operator's vision using a figure such that the operator can be easily informed of the amount of deviation between the position of the puncture needle N and the body surface puncture position. Each item of the position angle information and the mode of the figure may be appropriately interchanged. The figure representing the position angle information may be a figure other than the example shown in FIG. 7B or may be an indicator or the like.

In the ultrasonic laparoscopic puncturing system 2 of the second embodiment, an appropriate body surface puncture position can be obtained and the quality of an image displaying the body surface puncture position can be improved as in the ultrasonic laparoscopic puncturing system 1 of the first embodiment. In the ultrasonic laparoscopic puncturing system 2 of the second embodiment, the display device 40 is caused to display position angle information. Accordingly, the operator can ascertain a deviation between the needle tip position and the body surface puncture position and a deviation of the angle of the puncture needle N with respect to the puncture guideline while observing numerical values and figures displayed on the display device 40. Therefore, the puncture needle N can be punctured at an appropriate body surface puncture position at an appropriate angle.

In the ultrasonic laparoscopic puncturing system 2 of the second embodiment, the display control function 125 causes the display device 40 to display the amount of deviation according to position angle information. On the other hand, the display control function 125 may cause the display device 40 to display the position angle information using numerical values, figures, or the like, or cause the display device 40 to display the position angle information and the amount of deviation according to the position angle information.

### Third Embodiment

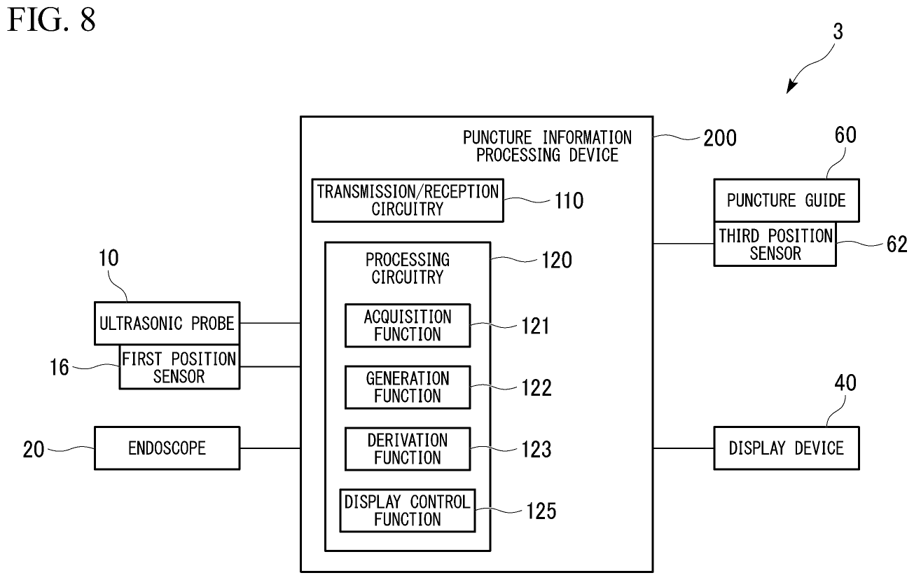

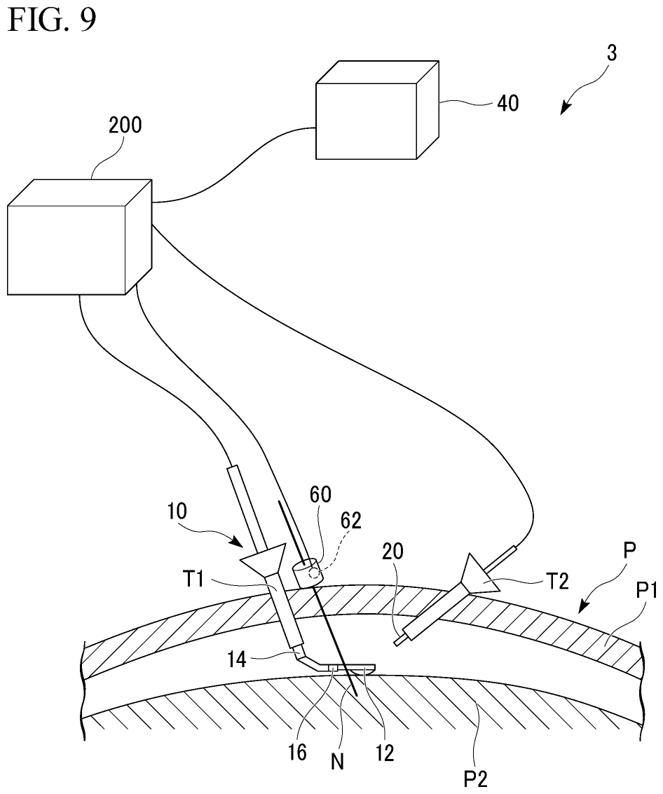

Next, a third embodiment will be described. FIG. 8 is a diagram showing an example of a configuration of an ultrasonic laparoscopic puncturing system 3 of the third embodiment and FIG. 9 is a cross-sectional view of a state in which an operation or the like is performed using the ultrasonic laparoscopic puncturing system 3. The ultrasonic laparoscopic puncturing system 3 of the third embodiment differs from the ultrasonic laparoscopic puncturing system 2 of the second embodiment in that the former includes a puncture guide 60 instead of the second position sensor 50, and a third position sensor 62 provided in the puncture guide 60. Hereinafter, the ultrasonic laparoscopic puncturing system 3 of the third embodiment will be described focusing on the differences from the ultrasonic laparoscopic puncturing system 2 of the second embodiment.

The puncture guide 60 is a cylindrical member. The puncture guide 60 has a through hole formed to penetrate the top surface and the bottom surface of the puncture guide 60, through which the puncture needle N penetrates. The bottom surface of the puncture guide 60 is placed on the body surface of the patient P during the operation. By using the puncture guide 60, the puncture needle N is stabilized when the puncture needle N is punctured. The puncture guide 60 is placed on the body surface of the patient P and guides the puncture position and the puncture angle of the puncture needle N. The puncture guide 60 may guide one of the puncture position and the puncture angle of the puncture needle N.

The puncture guide 60 has the third position sensor 62 built therein. The third position sensor 62 is, for example, a 6-axis sensor, and detects a position (a position of the puncture guide 60, hereinafter referred to as a “guide position”) and an angle (hereinafter referred to as a “guide angle”) of the exit portion of the through hole in the puncture guide 60. The third position sensor 62 may be a combination of, for example, a 3-axis sensor that detects accelerations in a three-dimensional direction and a 3-axis sensor that detects the angular velocity of each axis. The third position sensor 62 transmits the detected guide position and guide angle information to the puncture information processing device 200.

The acquisition function 121 in the processing circuitry 120 of the puncture information processing device 200 acquires information on the guide position and the guide angle transmitted from the third position sensor 62 and outputs the information to the derivation function 123. The derivation function 123 derives a body surface puncture position and a guideline angle and also derives an error between the guide position and the body surface puncture position and an error between the guide angle and the guideline angle.

The display control function 125 causes the display device 40 to display information on the error between the guide position and the body surface puncture position and the error between the guide angle and the guideline angle derived by the derivation function 123. For example, the display control function 125 causes the display device 40 to display the information on the error between the guide position and the body surface puncture position and the error between the guide angle and the guideline angle using numerical values or a figure as in the second embodiment in which the amount of deviation is displayed in the ultrasonic laparoscopic puncturing system 2.

In the ultrasonic laparoscopic puncturing system 3 of the third embodiment, an appropriate body surface puncture position can be obtained and the quality of an image displaying the body surface puncture position can be improved as in the ultrasonic laparoscopic puncturing system 1 of the first embodiment. In the ultrasonic laparoscopic puncturing system 3 of the third embodiment, the display device 40 is caused to display the error between the guide position and the body surface puncture position and the error between the guide angle and the guideline angle. Accordingly, the operator can ascertain the error between the guide position and the body surface puncture position and the error between the guide angle and the guideline angle while observing numerical values and figures displayed on the display device 40. Therefore, the puncture needle N can be punctured at an appropriate body surface puncture position at an appropriate angle.

In the ultrasonic laparoscopic puncturing system 2 of the second embodiment and the ultrasonic laparoscopic puncturing system 3 of the third embodiment, a body surface puncture position may be irradiated with laser light or an image of 3D mapping using the projection device 30 as in the ultrasonic laparoscopic puncturing system 1 of the first embodiment. Further, a guide path of the puncture needle N along a puncture guideline may be displayed in a three-dimensional space according to laser light or an image of 3D mapping using the projection device 30.

Although the puncture information processing device of each of the above embodiments is used in an ultrasonic laparoscopic puncturing system used in laparoscopic surgery, it may be used in other apparatuses. For example, it may be used as an inspection device for collecting and inspecting body fluids and cells from the body of a subject, and the like. Although the above-mentioned puncture information processing device derives a body surface puncture position and provides the operator with position angle information as information about the body surface puncture position, the derived body surface puncture position may be used in other aspect. For example, the body surface puncture position may be provided as a position at which a robot hand punctures a puncture needle at the time of performing treatment with the robot hand or the like.

According to at least one embodiment described above, it is possible to obtain an appropriate body surface puncture position by including an acquirer that acquires a passing position at which a passage through which a puncture needle for puncturing a living body passes is located when an ultrasonic probe provided with the passage is inserted into the living body, and a deriver that derives a body surface puncture position on a body surface to be punctured with the puncture needle passing through the passage on the basis of the passing position.

Although several embodiments have been described, these embodiments are presented as examples and are not intended to limit the scope of the invention. These embodiments can be implemented in various other forms, and various omissions, substitutions, and changes can be made without departing from the gist of the invention. These embodiments and modifications thereof are included in the scope and gist of the invention as well as in the scope of the invention described in the claims and the equivalent scope thereof.

What is claimed is:

1. A puncture information processing device, comprising processing circuitry configured to:
acquire position information on a passage through which a puncture needle for puncturing a living body passes when an ultrasonic probe provided with the passage is inserted into the living body; and
derive a body surface puncture position on a body surface of the living body to be punctured with the puncture needle passing through the passage on the basis of the acquired position information on the passage.

2. The puncture information processing device according to claim 1, wherein the processing circuitry is further configured to provide information on the body surface puncture position.

3. The puncture information processing device according to claim 2, wherein the processing circuitry is further configured to control a projector that projects light to project light to the body surface puncture position.

4. The puncture information processing device according to claim 2, wherein the processing circuitry is further configured to:
acquire position information on a needle tip of the puncture needle;
derive a relative relationship of a position of the needle tip with respect to the body surface puncture position; and
cause a display to display the relative relationship.

5. The puncture information processing device according to claim 4, wherein the processing circuitry is further configured to cause the display to display the relative relationship using numerical values or a figure.

6. The puncture information processing device according to claim 4, wherein the processing circuitry is further configured to:
derive a puncture angle at which the puncture needle punctures the body surface; and
cause the display to display information on the puncture angle of the puncture needle.

7. The puncture information processing device according to claim 6, wherein the processing circuitry is further configured to cause the display to display the puncture angle of the puncture needle using numerical values or a figure.

8. The puncture information processing device according to claim 1, wherein the processing circuitry is further configured to:
acquire position information on a puncture guide that is placed on the body surface and guides a puncture position of the puncture needle; and
derive an error between a position of the puncture guide and the body surface puncture position.

9. An ultrasonic laparoscopic puncturing system comprising:
a puncture needle that punctures a living body;
an ultrasonic probe provided with a passage through which the puncture needle passes;
a first position sensor that detects a position of the passage; and
the puncture information processing device according to claim 4,
wherein the processing circuitry is further configured to acquire a position of a needle tip of the puncture needle detected by the first position sensor.

10. An ultrasonic laparoscopic puncturing system comprising:
a puncture needle that punctures a living body;
an ultrasonic probe provided with a passage through which the puncture needle passes;
a first position sensor that detects a position of the passage;
a puncture guide that is placed on a body surface and guides at least one of a position and an angle of the puncture needle;
a second position sensor that detects position information of the puncture guide; and
the puncture information processing device according to claim 8,
wherein the processing circuitry is further configured to acquire the position information of the puncture guide detected by the second position sensor.

11. An ultrasonic laparoscopic puncturing system comprising:
a puncture needle that punctures a living body;
an ultrasonic probe provided with a passage through which the puncture needle passes;
a first position sensor that detects a position of the passage;
a projector that projects light; and
the puncture information processing device according to claim 8.

12. A puncture information processing method, using a computer, comprising:
acquiring position information on a passage through which a puncture needle for puncturing a living body passes when an ultrasonic probe provided with the passage is inserted into the living body;
deriving a body surface puncture position on a body surface of the living body to be punctured with the puncture needle passing through the passage on the basis of the acquired position information on the passage; and
providing information on the body surface puncture position.

13. A non-transitory computer-readable storage medium storing a program causing a computer to:
acquire position information on a passage through which a puncture needle for puncturing a living body passes when an ultrasonic probe provided with the passage is inserted into the living body;
derive a body surface puncture position on a body surface of the living body to be punctured with the puncture needle passing through the passage on the basis of the acquired position information on the passage; and
provide information on the body surface puncture position.
